(19) United States
(12) Patent Application Publication
(10) Pub. No.: US 2018/0190384 A1
(43) Pub. Date: Jul. 5, 2018

(54) AUTOMATED GENETIC TEST COUNSELING

(71) Applicant: Clear Genetics, Inc., San Anselmo, CA (US)

(72) Inventors: Mordechai Motti SHOHAT, San Anselmo, CA (US); Guy SNIR, San Anselmo, CA (US); Moran Shochat SNIR, San Anselmo, CA (US)

(21) Appl. No.: 15/863,641

(22) Filed: Jan. 5, 2018

Related U.S. Application Data
(60) Provisional application No. 62/442,586, filed on Jan. 5, 2017.
(60) Provisional application No. 62/488,479, filed on Apr. 21, 2017.

(51) Int. Cl. G16H 50/30; G16H 10/60; G06F 19/18

(52) U.S. Cl. CPC G16H 50/30; G06F 19/18; G16H 10/60

(57) ABSTRACT

Systems and methods here may be used to receive and analyze genetic risks based on patient data and analyze potential strategies to reduce risk. In some embodiments, systems and methods may be used to receive historical patient data and suggest pre-natal tests, receive results of the pre-natal tests and combine them with the historical patient data to make determinations for follow on tests as well as determine risk analyses for the patient. Finally, the systems and methods here may be used to display the results of the test data and risk analysis in graphical user interfaces that may be easily analyzed by a healthcare provider.

### CROSS REFERENCE TO RELATED CASES

This application is related to and claims priority to U.S. Provisional 62/442,586 filed 5 Jan. 2017 and U.S. Provisional 62/488,479, filed on 21 Apr. 2017 which are both hereby incorporated by reference in their entirety.

### TECHNICAL FIELD

This application relates to the field of networking, analytics, and automation of genetic testing communications.

### BACKGROUND

Advances in technology and genomics are boosting availability, awareness, and usage of genetic tests, generating more information about human genetic disorders than ever before. According to the National Center for Biotechnology Information (NCBI), there are over 48,700 genetic tests available today, screening for over 10,500 conditions. Accompanying this surge in test supply is a severe shortage in resources that can advise on genetic testing.

The current landscape of genetic testing is messy. Patients are given a wide range of testing options without given any parameters that may apply to them or their family history. Without a framework for organizing and automating genetic test counseling, healthcare providers are left without direction as to how to determine which genetic tests to apply and which to avoid. Patients are given general information that is not tailored to their history and circumstances because individual medical personnel are incapable of processing the amount and kinds of information needed to then make an appropriate assessment.

It is assumed that 40% of the tests performed are ordered incorrectly, as doctors struggle to provide their patients with answers. Some statistics show that 1 in 33 children are born with some type of severe genetic disease. Genetic diseases are a leading cause of infant death and the estimated cost to the healthcare system is more than $50B annually.

### SUMMARY

Systems and methods here may be used to evaluate genetic risks based on patient data and analyze potential strategies to reduce risk. In some embodiments, systems and methods may be used to receive historical patient data and suggest pre-natal tests, receive results of the pre-natal tests and combine them with the historical patient data to make determinations for follow on tests as well as determine risk analyses for the patient. Finally, the systems and methods here may be used to display the results of the test data and risk analysis in graphical user interfaces that may be easily analyzed by a healthcare provider.

### BRIEF DESCRIPTION OF THE DRAWINGS

FIG. 1 is a strategy flow chart according to certain embodiments disclosed here.

FIG. 2 is a network diagram used to practice the methods according to certain embodiments disclosed here.

FIG. 3 is a graphical user interface (GUI) example customer interface page according to certain embodiments disclosed here.

FIG. 4 is a GUI example test results page according to certain embodiments disclosed here.

FIG. 5 is another GUI example test results page according to certain embodiments disclosed here.

FIG. 6 is another GUI example test results page according to certain embodiments disclosed here.

FIG. 7 is another GUI example test results page according to certain embodiments disclosed here.

FIG. 8 is another GUI example test results page according to certain embodiments disclosed here.

FIG. 9 is a diagram of computer hardware used to practice the certain embodiments disclosed here.

### DETAILED DESCRIPTION

Reference will now be made in detail to embodiments, examples of which are illustrated in the accompanying drawings. In the following detailed description, numerous specific details are set forth in order to provide a sufficient understanding of the subject matter presented herein. But it will be apparent to one of ordinary skill in the art that the subject matter may be practiced without these specific details. Moreover, the particular embodiments described herein are provided by way of example and should not be used to limit the scope of the invention to these particular embodiments. In other instances, well-known data structures, timing protocols, software operations, procedures, and components have not been described in detail so as not to unnecessarily obscure aspects of the embodiments of the invention.

Overview

Systems and methods here provide tools to a healthcare provider such as an obstetrician to help determine what the appropriate pre-natal testing selection should be based upon. The analysis uses historical genetic and medical data of the parent(s) to suggest follow-on tests. The results of these follow-on tests, together with the historical genetic and medical data may be used to suggest personalized genetic syndromes risk estimations and also additional tests and their implications.

The medical basis on which the suggestions are based form the analytics which provide options for follow on testing and information, customized to each particular patient. For example, researchers have studied the risk for Down syndrome and other chromosomal anomalies according to parental age, what is the effect of almost every finding on the likelihood ration of these syndromes, and the lowering effect of the different tests on these risks. By personalizing treatment, based on gathered and analyzed data, the healthcare provider may be able to deliver customized, accurate, and whole care to patients, instead of delivering a generic set of information to all patients. This may empower patients and healthcare providers to tailor treatment plans before, during and after pregnancies.

These systems and methods may include gathering historical and testing data, analyzing the data to calculate various risks, and suggested follow on testing. Various analytics may be presented in reports of personalized information that is actionable and clear for healthcare providers to use to tailor treatment plans for individual patients.

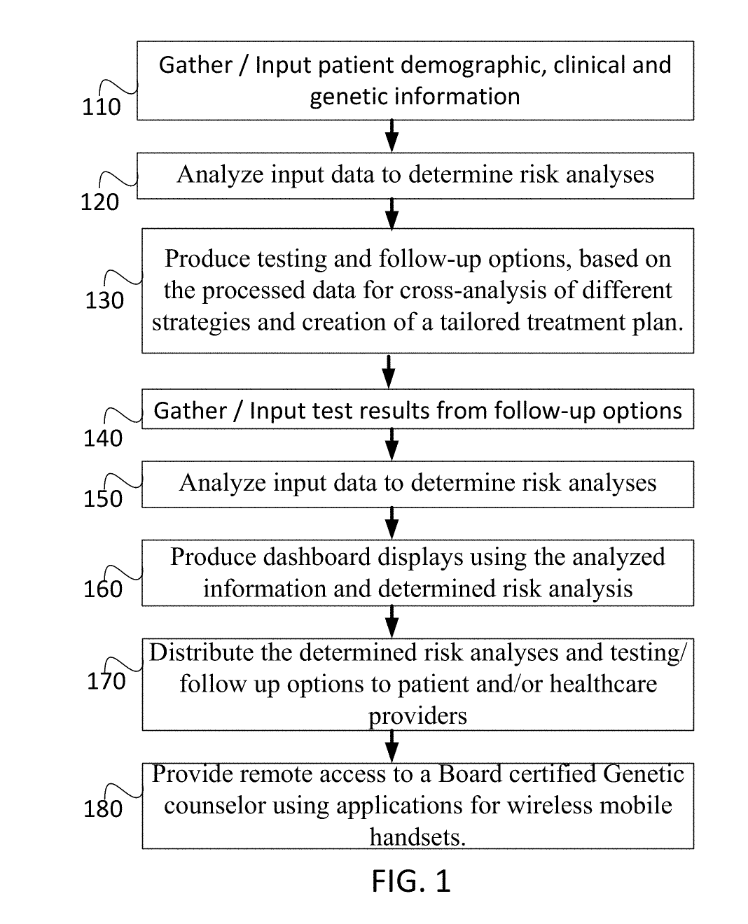

FIG. 1 shows an example high level process which may be used as described herein, at various intervals during a normal pregnancy. FIG. 1 at 110 shows step 1—Gather and/or input patient historical information into the systems for analysis and processing. This information may include parental information such as demographic, clinical, medically historical information and genetic information. FIG. 1 at 120 shows step 2—Back end systems may then take and analyze the input historical patient data to determine various risk analyses. FIG. 1 at 130 shows step 3—Back end systems may produce testing and follow-up options, based on the processed testing data for cross-analysis of different strategies and creation of a tailored treatment plan. FIG. 1 at 140 shows step 4—Gather and/or input patient test data from the testing and follow-up options from Step 3 106. FIG. 1 at 150 shows step 5—Back end systems may then take and analyze the input test data to determine various risk analyses. FIG. 1 at 160 shows step 6—Produce dashboard displays using the analyzed information and determined risk analyses. FIG. 1 at 170 shows step 7—Distribute the determined risk analyses and testing and/or follow up options to patient or healthcare providers. FIG. 1 at 180 shows step 8—Telegenetics—Provide remote access to a Board certified Genetic counselor using applications for wireless mobile handsets.

Networked Systems

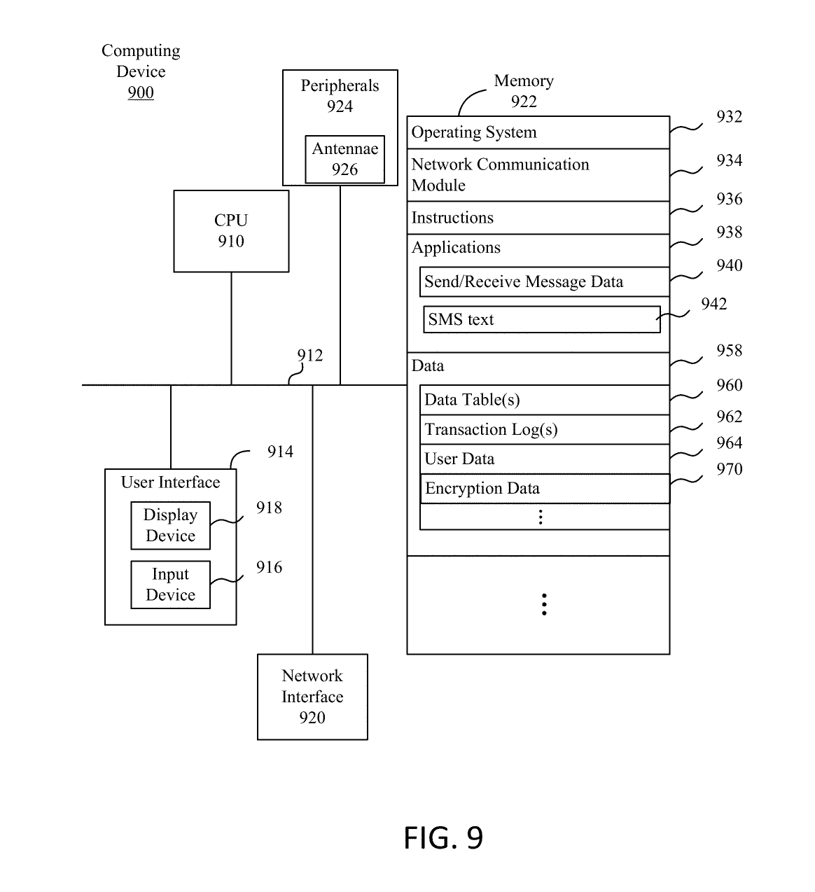

The systems and methods here may be carried out using many various computers including but not limited to networked computers, databases, server computers and displays including but not limited to wireless handheld devices such as smartphones and tablet computers. Details of such computers are described in detail in FIG. 9.

In some embodiments, calculations and displays may be conducted at the local handheld device level. In some embodiments, back end systems may be used for more complex or memory intensive analysis, and/or data storage and results sent to local wireless devices. In some examples, a blend of local and back end computations and data storage may be used to provide efficient and fast computation and graphically rich user interfaces.

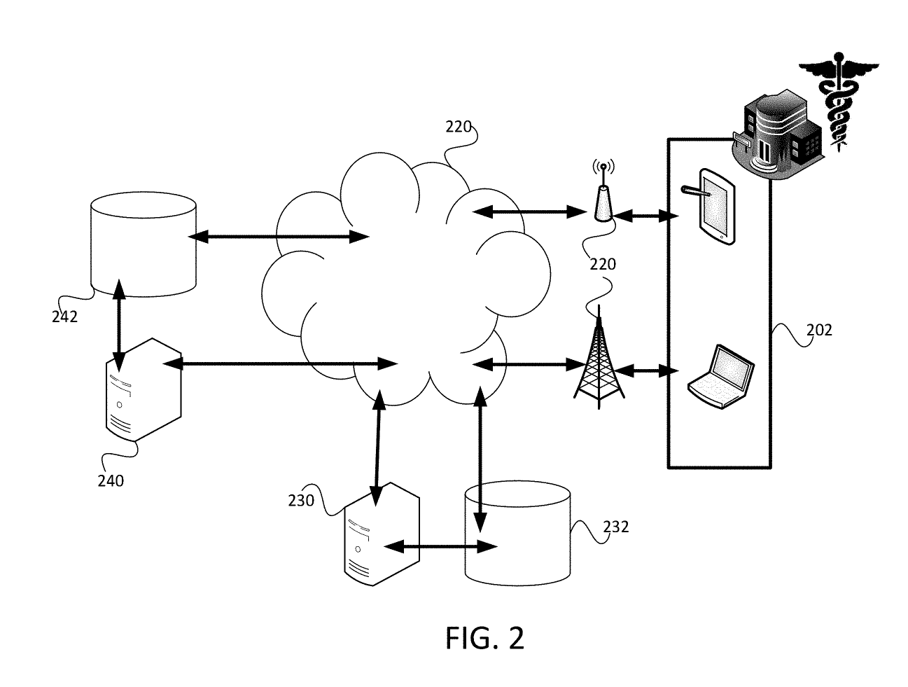

FIG. 2 shows an example of a network arrangement of computing systems which the systems here may employ to carry out their methods. FIG. 2 shows example user devices 202 for example, any kind of computing device such as a wireless smartphone, laptop, tablet computer, or wired desktop computer. User devices 202 may also be wearable computers such as glasses, watches, heart monitors, blood pressure monitors, cuffs, bracelets, necklaces, or other peripheral wearable. Such user devices may have any number of computer components such as those found in FIG. 9.

In the example embodiment in FIG. 2, user devices 202 may be able to interface with a network 220 such as the internet by way of any of various systems such as wireless radios, cellular systems, or other communication systems 210. In the example, the user devices 202 can utilize Wi-Fi, Bluetooth Low Energy, and/or cellular communication methods to communicate by way of the internet 220. The example of FIG. 2 also shows any of various back end systems such as computer servers 130 and data storage 232 which may be in communication with and able to be communicated with by the internet 220. Such systems may include any number of server computers 230 able to send, receive, process and analyze data according to the descriptions here. The server computers 230 may be able to cause storage of data in data storage systems 232 that are local, distributed, networked, or any combination of these. The back end server systems may also be distributed with various server computers 240 physically located remotely from others 230 but able to communicate through the internet or proprietary network 220 with one another and with the various user devices 202. Additionally or alternatively, any of various cloud or networked storage could be used to access data storage 232, 242 located locally to the server systems 230, 240 or remotely through a network such as the internet 220.

By using such systems, the various testing data may be input, transmitted, analyzed and downloaded to the resources that can most efficiently complete the required tasks. In some embodiments, data storage and/or data processing may be done locally on the user device itself 202 by software applications running on the user device itself 202. In some embodiments, data is sent to any of various back end servers 230, 240 for processing and data sent to storage 232, 242 for storage. In some embodiments, a combination of local and back end systems may work together to efficiently process and store data.

Chat Functionality

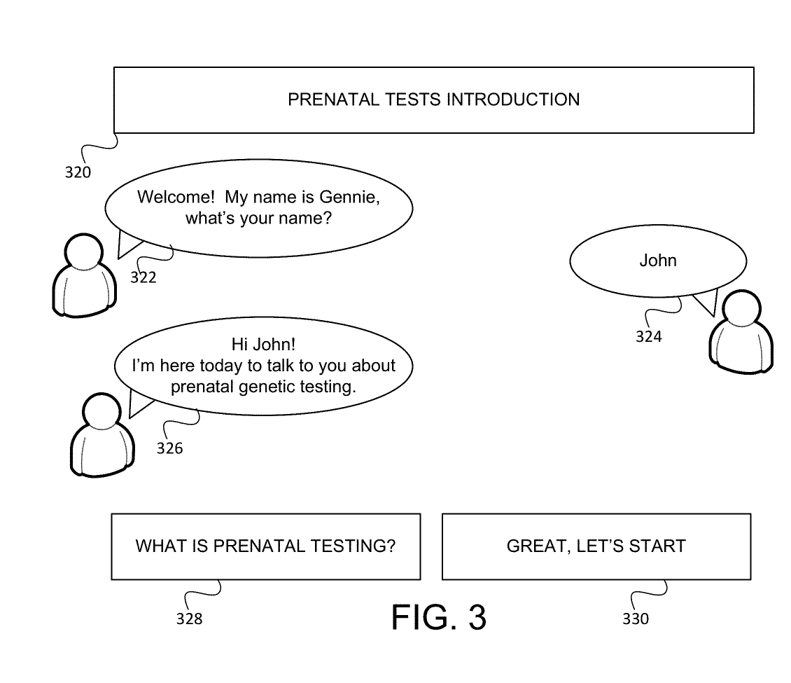

The systems described here may be used to interact with users by way of the internet as described in FIG. 2. FIG. 3 shows an example GUI screen with interactions depicted between an automated chat BOT and a human customer user. In the example, the system is designed to receive and answer questions from the human user by itself, without input from a user in real time. The system is able to receive and analyze questions, provide relevant answers, and communicate back and forth with users. The advantages of such a system are to alleviate the need for human interaction to answer standard questions on the topics discussed here. Using analysis of the text questions, answers may be provided by the system over the network.

The example of FIG. 3 shows an example graphical user interface (GUI) screen with a Prenatal Tests Introduction banner 320. The interfaces could be used in any part of the process, by the system to interact with a user. In this example, an introduction is shown, but could be any kind of GUI and/or interface.

The system represents itself to a user by interfacing as if a person were chatting with the user. In the example, the computer, also called a “bot” or “BOT,” introduces itself as if it were a person 322. Next, the human user responds by answering the questions posed to him or her. In the example, the question is what is the user's name. 324 The system is able to interpret the response to the question by analyzing the text response. In some examples, the system uses optical character recognition to analyze the responses received by the system over the network. In some example embodiments, the computer bot is able to follow a script preloaded by the system administrator to interact with users. The system may then compare the received responses to a template, table, list, or other database which may provide a follow on question, while storing the received data. Some examples utilize artificial intelligence, machine learning, or other computer software in order to receive and interpret answers before sending follow on questions or provide answers. In such examples, the computer bot may be able to react to questions by the user, receive answers to questions that it asks a user, and analyze those answers to produce meaningful answers for the user. The system may thereby use the information when introducing the topic of the GUI interface. 326 The system is thereby able to answer many kinds of questions and analyze the answers in order to arrive at a customized solution for the users.

The example of FIG. 3 also shows two buttons at the bottom of the GUI interface, the first button is labeled What is Prenatal Testing 328. This button allows a user to receive answers to previously scripted questions without having to interface with the bot. The second example button is labeled Great, Lets Get Started 330. This button allows the user to move off of the bot GUI interface screen and begin the information input which is used in the other portions of the system to suggest testing. The examples of using two buttons with these functionalities is not intended to be limiting. Any number of previously scripted questions and answers may be utilized on the GUI. Additionally or alternatively, any number of page navigation buttons may be used to navigate off of the initial introduction GUI screen.

In some example embodiments, the user is able to input all of the required information within the bot question and answer formatted screen. In such examples, the system bot provides questions and the user inputs answers which are received by the system and stored. In such examples, the system may be able to conduct a preliminary analysis on the input answers from the user, and analyze them for a deficiency. For example, if the system bot requests a name and the user inputs a phone number, the bot system may receive the phone number as an answer and analyze it to determine that no letters are present in the answer, only numbers. In such examples, the bot system may prompt the user to input the answer to the same question, until certain parameters are met for the answer.

In some example embodiments, the system may navigate the user to another page which is used as a separate input interface. In such examples, a table or series of input boxes may be used to prompt the user to input the appropriate information. In such examples, a the bot system may only be used for sending and receiving answers to general questions on the bot question and answer screen.

Security Examples

The systems used to analyze the testing data and provide test and follow-up options may include various levels of security due to the nature of the information being gathered, analyzed, transported, and stored. In some embodiments, encryption of the test data may take place before any test data is moved or processed. In some embodiments, encryption may take place before storage of any test data. Alternatively or additionally, user information may be encrypted as well. In some examples, government regulations may provide guidelines on how such data is handled, such as Health Insurance Portability and Accountability Act of 1996 (HIP2) guidelines.

In one example, a web page may be used to interface with users to input data and view results. Such users may be patients and/or healthcare providers, depending on the interface. Various forms of security may be used to access such systems and information. For example, personal identification numbers could be assigned to practitioners who are allowed access by a system administrator. In some examples, a login and password combination may be used. In some examples, a cycling security number may be provided on a mobile application or remote device. In some examples, biometrics such as fingerprints or retina or iris scans could be used. Any of various security measures could be arranged to safeguard access to the underlying test data and the user information as well as access to the systems themselves.

Analysis Input

The underlying analysis that the systems here may undertake can include two sources of data in order to customize results and suggestions. The first of the groups is personal historical information. The second is individual test data.

Information Input—Personal Historical Information

In examples that utilize the first group of data, personal historical information or data, the healthcare provider may gather and input the data into the system for storage and analysis. For example, once the healthcare provider user gains access to the system, various historical information can be uploaded for correlation to a particular patient. For example, identifying information for a patient could be uploaded to ensure each patient record is separately stored and treated. Additional information such as family history, genetic history, medical history could also be uploaded to the system. Input and storage of such information may be conducted through the computer systems as described in FIG. 2 and FIG. 9.

Such information could be uploaded through selection of various interfaces in the system for each patient. For example, the system could prompt the user to select whether the patient is a diabetic or not, through a drop down or radio button selection. Any kind of risk identifiers and medical history questions could be asked of each patient. Additionally or alternatively, demographic information could be input into the system such as age of a patient including, but not limited to the following: whether the maternal age is greater than 35 years at the time of the last menstruation period, race, creed, domicile, education level, class, or other demographics. Again, the information could be entered through field inputs and/or preselected drop down menus.

Medical history information may also be input to the system and correlated to the file of a patient. Such medical history may include but is not limited to: family history of genetic diseases, previous pregnancies outcome, ethnicity/inbreeding coefficient, medications/teratogenes, date of last menstrual cycle, number of previous children, number of previous complicated births, medications taken which increase the risk for chromosomal anomalies, whether parents are carriers for a mendelian disease that can be detected pre-natally, one of the parents with a balanced translocation, previous pregnancy to the couple with a documented chromosomal anomaly, or any other kind of personal information such as previous medical conditions. Again, the information could be entered through field inputs and/or preselected drop down menus.

In some embodiments, the system may then analyze the input historical patient information a first time in order to determine a risk analysis. The system may then compare the risk analysis to a database to recommend follow on tests. In some embodiments, this first analysis may be combined with an analysis of some testing results as well.

Information Input—Test Data

Once the patient file is saved on the system with all of the history and demographic information entered, the results of any tests may also be entered and correlated to the same patient files. This test data could be from tests conducted in this or any other pregnancy. Each test may include its own set of result inputs, depending on the type of test and the kind of data that is returned from each test result. Again the input of the test results could be field entries into the webpage or application, or drop downs of previously loaded result possibilities. The dates of each test could be input and the various tests could each include its own page or field or combination. The test data may be updated and refreshed as new tests are run and test results become available. Testing history may be gathered by the system in this way, including historical test data based on test dates.

Example tests include but are not limited to carrier screening test results for inherited conditions, number of fetuses, nuchal translucency, biochemical markers, ultrasound findings (markers as well as major anomalies/findings), fetal growth parameters as well as parental growth parameters, and other tests. Examples of basic prenatal screening tests may include but are not limited to ultrasound scans, alpha-fetoprotein tests, chorionic callus sampling, amniocentesis, percutaneous umbilical blood sampling.

Other example tests which may be used to gather information include but are not limited to glucose tolerance tests, fetal non-stress tests, biophysical profiles, triple screen tests: to measure low and high levels of AFP, abnormal levels of hCG and estriol, quad screen tests: to measure alpha-fetoprotein (AFP), human chorionic gonadotropin (hCG), Estriol, and Inhibin-A, tests for PAPP-A, Chorionic Villus Sampling (CVS), a urinalysis to analyze blood type, Rh factor, glucose, iron and hemoglobin levels.

DNA and other paternity tests may also be conducted, blood tests for Down Syndrome Trisomy-21 and Trisomy-18 as well as nuchal translucency (fluid beneath the skin behind baby's neck) tests.

Other tests may be used to determine non-chromosomal severe bad outcomes due to neuro-genetic syndrome or bad neurologic outcome. In such examples, the estimated combined statistical risks for various syndromes associated with intellectual disability and other severe neurological bad outcome that cannot be detected in any of the standard pregnancy follow up, and even with normal detailed ultrasounds and normal in-depth chromosomal evaluation of the amniotic fluid.

Test Result Analysis

After the patient information and test information is entered into the system, the system can then process the information in order to determine a customized risk analysis profile for any given patient. In some embodiments, the risk could be manipulated manually by the physician or counselor reviewing the dashboard with the patient, to account for specific consideration that make a case unique.

The risk analysis could be run for any of various maladies and conditions including but not limited to Down's syndrome and chromosomal anomalies as well as other neuro-genetic malicious outcomes. The risk analysis could be used to determine whether any more follow-on tests are desired, needed, or are optional.

The system may be used to generate various kinds of results. One example result is a likelihood ratio to incorporate results based on all of the factors discussed. The analysis may also be used to pinpoint vulnerabilities in patient test results, examine genetic tests according to impact, unify recommendations and align with guidelines, and even simplify administration of records by combining test results into one system.

The system may utilized historical testing data in various ways in its analysis. For example, one analysis may utilize all of the historical test data for one particular test, but only the latest test result from a series of another test. As new test data is entered, the analysis may be updated and re-calculated.

The system may also analyze data for chromosomal anomalies, for example, the estimated combined statistical risks for Down syndrome, the estimated combined statistical risks for other chromosomal anomalies that can be identified in the amniotic fluid by standard karyotyping, the estimated combined statistical risks for chromosomal anomalies that can be detected in the amniotic fluid by using new technology such as prenatal chromosomal microarrays (CMA).

The system may be used to determine how each risk changes with several tests such as but not limited to: noninvasive prenatal testing (NIPT tests), Amniocentesis with CMA analysis and Normal follow up.

The analysis may yield customized likelihood ratios which may indicate an increased or decreased risk for a specific condition that is calculated if the finding is marked. These results may be integrated—for example, if two independent findings which increase the risk for Down syndrome are found, each with a likelihood ration of three, the total increase in Down syndrome result is weighted as a nine. Some embodiments use assumptions that the tests are not correlated. This may or may not be likely, but such assumptions may be used when no better data is available.

In the program, for each finding such as medical history, abnormal result, or any abnormality, its potential influence is calculated with the likelihood ratio on each of the various diseases/conditions for which the system is asked to calculate the integrated risk. Tables or databases may be used to correlate findings, and whether among them, if they are dependent upon one another. If two findings are dependent, there may be a specific rule used when analyzing the combinations. The amount of risk reduction by the different test may also be known.

Based on the calculations above, the system may make recommendations for follow on care and particular suite of tests. Example of follow on tests may include but are not limited to panel carrier testing for known mutations for inherited conditions. These may be mostly autosomal recessive inherited common genetic diseases such as exome sequencing of the fetal DNA from the amniotic fluid as well as the parents. Other specific tests may be based on the individual family history or pregnancy's test results per the geneticist recommendation. Further, the risk for other conditions such as specific diseases associated with the specific findings may be determined. Extra risks for obstetrical problems associated with the pregnancy findings. Based on this the program categorize the degree of indication for: Genetic counseling; Amniocentesis or NIPT; other specific tests.

Results Display Examples

Once the tests are analyzed and results are determined, the systems and methods here may be used to cause display of graphical user interfaces (GUIs) of the results so healthcare providers may clearly understand the analysis. For example, the system may combine estimated risks and plot the results on a schematic graph in comparison to the mean risk of the general population at the same maternal age. Such GUIs may be presented through a webpage portal, through an application which may be loaded on a mobile device such as a smartphone or tablet computer, such information may be printed on a PDF or Word document for the client.

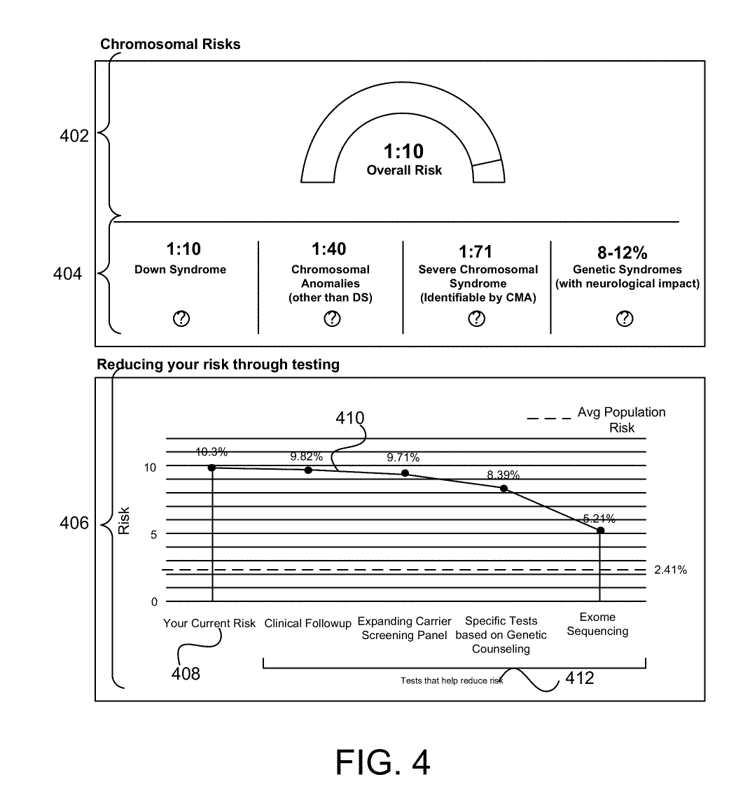

FIG. 4 is an example result GUI showing Chromosomal Risks using an overall risk rainbow chart 402 as well as breaking down more of the results into graphics with likelihood rations 404. The example of FIG. 4 also shows a graph 406 depicting a Reduction of Risk Through Testing. Such a graph includes the patient's current risk determination 408 graphed against potential risk analysis if that patient undergoes various other tests. The graph shows 410 the reduction in risk for each of the various tests that may help reduce or better understand the risk 412.

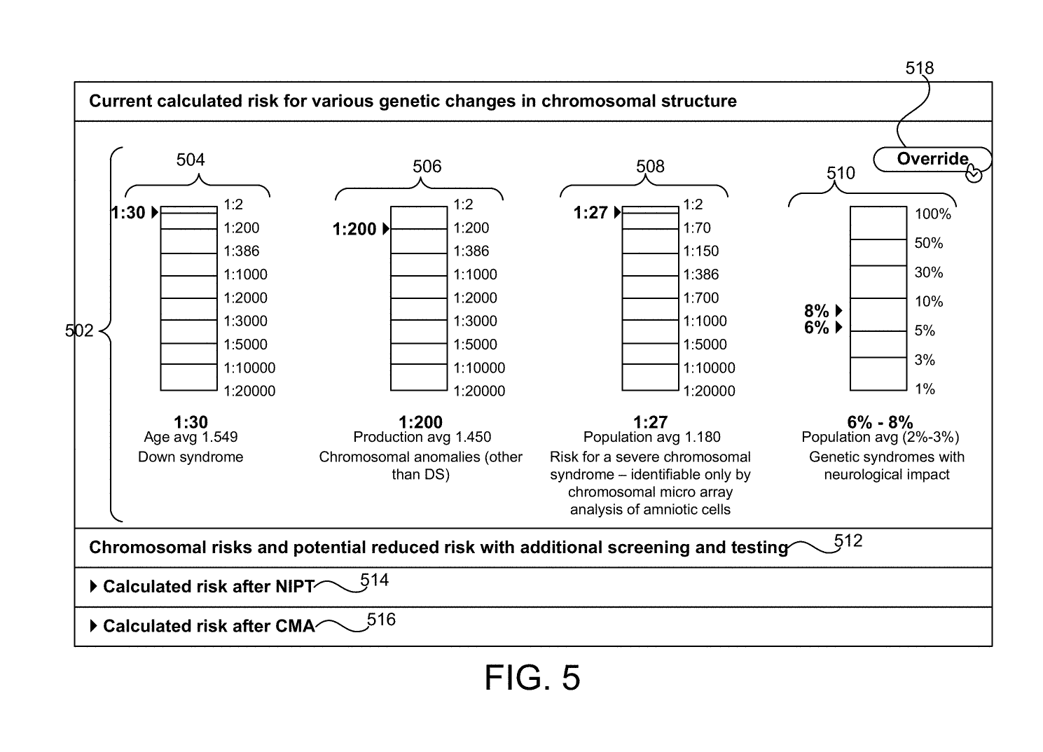

FIG. 5 is an example result GUI showing Current Calculated Risk for Various Genetic Changes in Chromosomal Structure. The main screen section 502 show four representative graphs 504, 506, 508 and 510. These bar graphs show various test results based against some kind of average. For example, 504 shows a patient's results for Down's Syndrome as 1 in 30 based against an average for the subject's age at 1 in 549. Graph 506, shows a patient's results for Chromosomal anomalies other than DS at 1 in 200 based on an average of 1 in 450. Graph 508 shows a patient's results for severe chromosomal syndrome at 1 in 27 based on a population average of 1 in 180. Finally, FIG. 5 shows a GUI of graph 510 which shows this subject's risk of genetic syndromes with neurological impact at between 6%-8% as compared to a population average of 2%-3%.

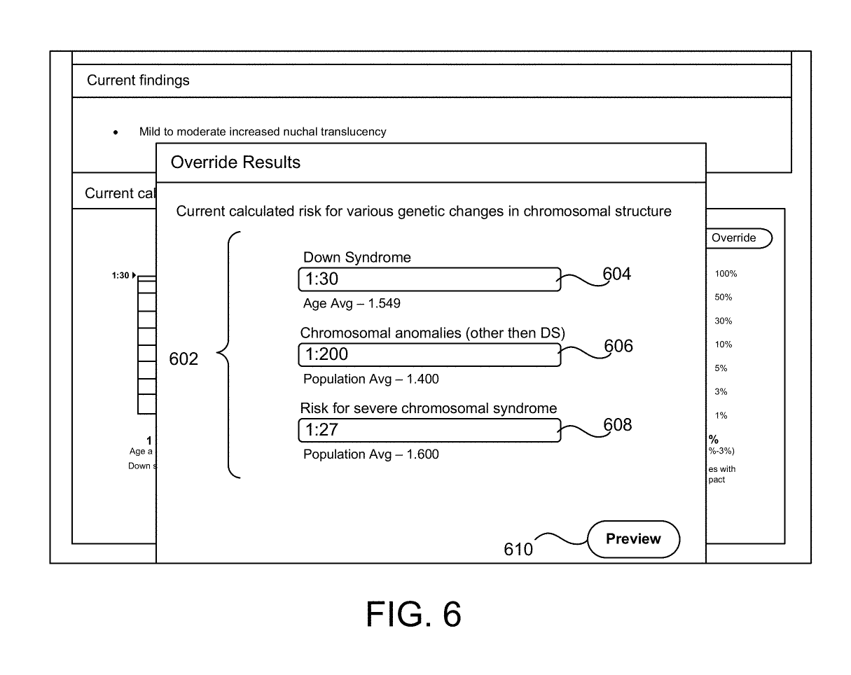

The GUI of FIG. 5 also shows options for a user to select Chromosomal risks and potential reduced risk with additional screening or testing 512. This selection could navigate the user to the GUI of FIG. 4 as described herein. Further, the user may be able to select Calculated risk after non-invasive prenatal testing (NIPT) 514 and Calculated risk after chromosomal microarray (CMA) 516. Such selections may change the four graph results 504, 506, 508 and/or 510 accordingly. Further, FIG. 5 shown an Override button 518 which may be selected by a user. Such a selection may navigate the user to the GUI as shown in FIG. 6. In some embodiments, the Override may allow a medical professional facilitating the discussion to change the risk based on judgement, thus overriding some portion of the results.

FIG. 6 shows an example GUI of an Override Results page 602. Such an override results page 602 is shown as a popup in the GUI of the results page of FIG. 5 but could be a separate window or other embodiment. The override results page may be displayed when the user selects the Override button in the previous screen as shown in FIG. 5. In the example Override Results page, the system displays current calculated risk for various genetic changes in chromosomal structure. In the example of FIG. 6, the system shows Down Syndrome 604, Chromosomal anomalies other than DS 606 and Risk for severe chromosomal syndrome 608.

In such examples, an overlay opens when the overlay button is clicked for a form section. In some embodiments, current values are pre-populated into the fields. Average values may be displayed as a reference. And in some GUI examples, a preview button implies the results are not saved as a derivative report.

FIG. 6 also shows a Preview button 610 which in some embodiments may allow the system to present a preview of the input change in the rest of the report.

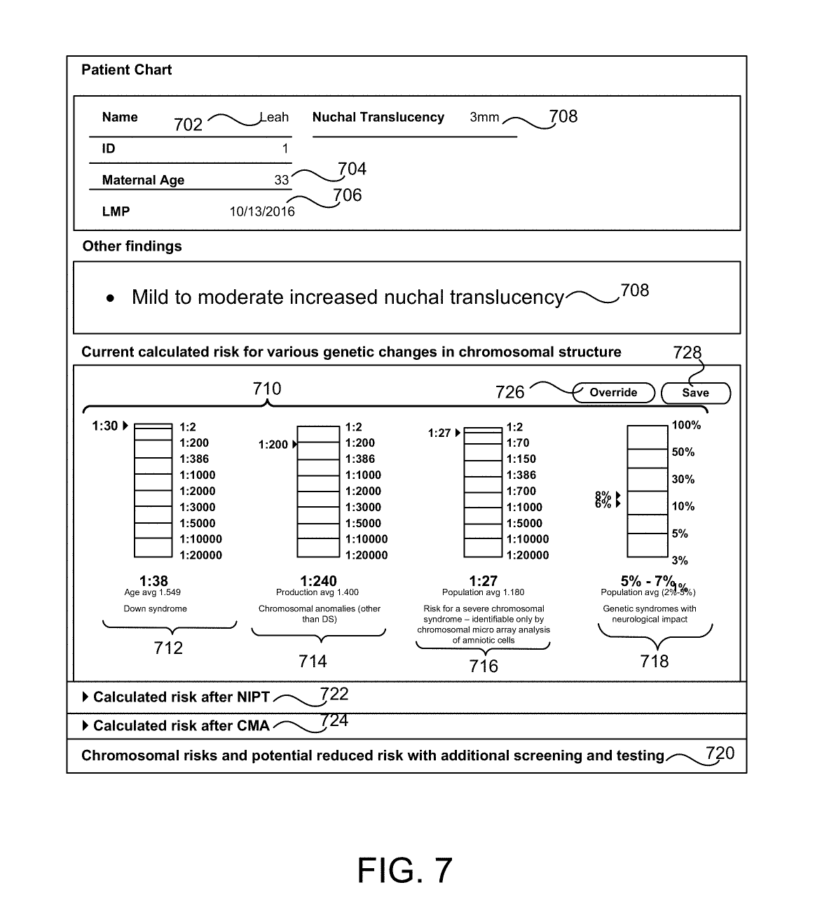

FIG. 7 shows an example GUI of a Patient Chart, which may be used as an interface for the healthcare provider to easily review test results and analysis for a particular patient. The example patient chart FIG. 7 shows the name of the patient 702, age 704 and date of last menstrual period 706. The example GUI also shows the test results of one test, in this example, nuchal translucency 708. The example of FIG. 7 also shows other finding section 710 where the system may push information relevant to the test result 708 or may further explain testing options.

The example GUI of FIG. 7 also shows the data from FIG. 5 with the current calculated risk for various genetic changes in chromosomal structure 710 with four graphs including in this example, Downs Syndrome 712, chromosomal abnormalities besides DS 714, risk for severe chromosomal syndrome 716, generic syndromes with neurological impact 718. And as shown in FIG. 5, the option is available to show the calculated risks and potential reduced risk with additional screening and testing 720 after NIPT 722 and after CMA 724.

As explained in FIG. 6, the FIG. 7 also includes an override button 726 and a Save button 728. The Save button 728 may be used to save the charts and graphs of a GUI after the healthcare provider has changed some of the results.

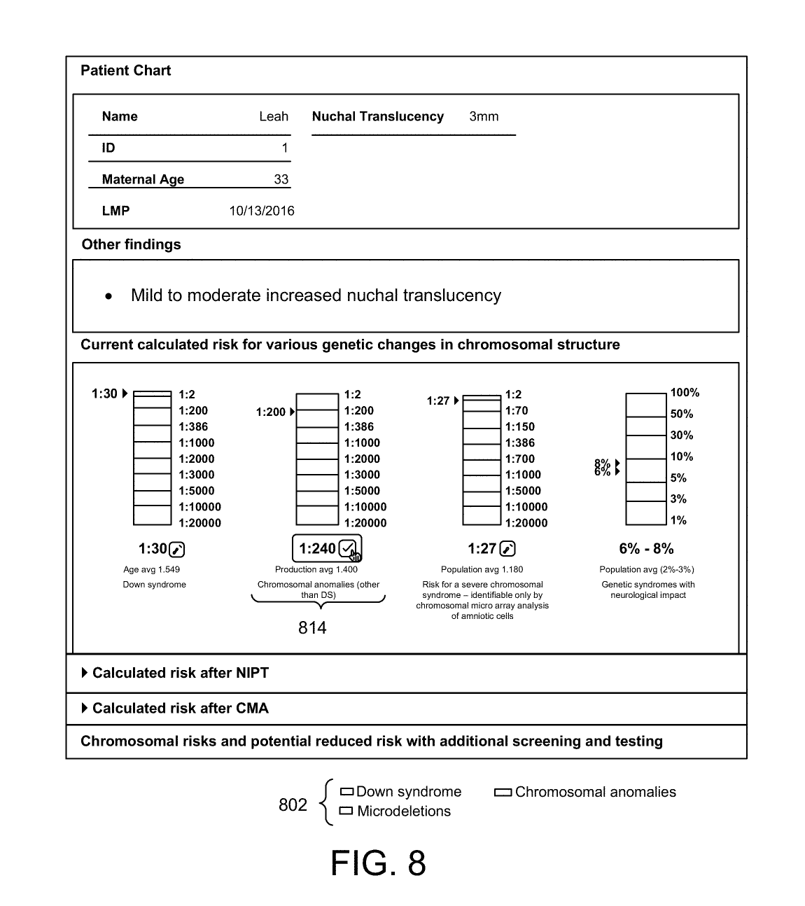

FIG. 8 shows the GUI of FIG. 7 but when the user selects one of the graphs 814. By selecting one of the graphs, the user may then gain more understating of the underlying data used in that specific determination. The GUI of FIG. 8 also allows the user to select a specific malady 802 to learn more details on the test results for the patient.

Coverage

The systems and methods here may also provide administrative information regarding the coverage of each test based on the risk and effectiveness of the test according to the specific couple/results. Insurance coverage can be adjusted to the specific rules of the HMO or state. For example, in Israel the coverage for amniocentesis is split between ministry of health, HMO, complementary insurance, private insurance or out of pocket—and it is according to the cause for the risk (for example: maternal age, ultrasound findings by the ministry and biochemical marker screening is paid by the HMO, etc.)—which may be conducted automatically, using the systems and methods here which are accepted by the public and save administrative work.

Example Down Syndrome Estimation Examples:

Below are examples of Down Syndrome test analysis and results conclusions. These examples are not intended to be limiting but merely exemplary and could be used in any combination.

In some examples, the calculation to incorporate the test results used here have been based on the accepted medical recommendations, according to the general recommendations of the American as well as the Isreali Societies of Medical Genetics and according to the relevant medical literature, in the manner described herein:

In some examples, the most accurate estimation of the Down Syndrome risk which combines results of first and second trimester Down Syndrome screenings including nuchal translucency, maternal serum biochemical markers in the first and second trimesters has been based on the recommendation of the Society of Medical Genetics.

The influence of the soft ultrasound markers on the Down syndrome screening calculated risk has been determined according to the literature. There are at least two ways currently in use for such a task.

Likelihood ratios for various “soft markers” for Down syndrome as isolated markers with second trimester genetic sonography. Likelihood ratios for various “soft markers” for Down syndrome, regardless of whether isolated or multiple, with second trimester genetic sonography.

Among these two strategies, some embodiments use Nyberg's suggestions with modified factors to those commonly used by most genetic counselors. Although the original reports include a reduction in the Down syndrome risk if no ultrasound findings have been identified, this embodiment does not include that aspect, as many genetic counselors do not yet do that in common practice.

When the nuchal translucency (NT) is given in size and no data is filled for the NT based Down syndrome risk, the systems and methods here calculate the NT based risk assuming the woman is eleven (11) weeks gestation at the time NT was measured (that allows more conservative calculation).

In some embodiments, a combination of first and second trimester screening results may include calculations of the combined risk once assuming no relationship and once assuming there is a relationship between the two results. The greater risk between the two results is chosen as the integrated risk.

A significant ultrasound finding such as: a major anomaly, a nuchal translucency greater than 3 mm at 11-13 weeks gestation, more than two soft signs by ultrasound.

Example Computing Device

FIG. 9 shows an example computing device 900 that may be used in practicing certain example embodiments described herein. Such computing device 900 may be the back end server systems use to interface with the network, receive and analyzed data, as well as generate test result GUIs. Such computer 900 may be a mobile device used to create and send in data, as well as receive and cause display of GUIs representing data. In FIG. 9, the computing device could be a smartphone, a laptop, tablet computer, server computer, or any other kind of computing device. The example shows a processor CPU 910 which could be any number of processors in communication via a bus 912 or other communication with a user interface 914. The user interface 914 could include any number of display devices 918 such as a screen. The user interface also includes an input such as a touchscreen, keyboard, mouse, pointer, buttons or other input devices. Also included is a network interface 920 which may be used to interface with any wireless or wired network in order to transmit and receive data. Such an interface may allow for a smartphone, for example, to interface a cellular network and/or WiFi network and thereby the Internet. The example computing device 900 also shows peripherals 924 which could include any number of other additional features such as but not limited to an antennae 926 for communicating wirelessly such as over cellular, WiFi, NFC, Bluetooth, infrared, or any combination of these or other wireless communications. The computing device 900 also includes a memory 922 which includes any number of operations executable by the processor 910. The memory in FIG. 9 shows an operating system 932, network communication module 934, instructions for other tasks 938 and applications 938 such as send/receive message data 940 and/or SMS text message applications 942. Also included in the example is for data storage 958. Such data storage may include data tables 960, transaction logs 962, user data 964 and/or encryption data 970.

### CONCLUSION

As disclosed herein, features consistent with the present inventions may be implemented by computer-hardware, software and/or firmware. For example, the systems and methods disclosed herein may be embodied in various forms including, for example, a data processor, such as a computer that also includes a database, digital electronic circuitry, firmware, software, computer networks, servers, or in combinations of them. Further, while some of the disclosed implementations describe specific hardware components, systems and methods consistent with the innovations herein may be implemented with any combination of hardware, software and/or firmware. Moreover, the above-noted features and other aspects and principles of the innovations herein may be implemented in various environments. Such environments and related applications may be specially constructed for performing the various routines, processes and/or operations according to the invention or they may include a general-purpose computer or computing platform selectively activated or reconfigured by code to provide the necessary functionality. The processes disclosed herein are not inherently related to any particular computer, network, architecture, environment, or other apparatus, and may be implemented by a suitable combination of hardware, software, and/or firmware. For example, various general-purpose machines may be used with programs written in accordance with teachings of the invention, or it may be more convenient to construct a specialized apparatus or system to perform the required methods and techniques.

Aspects of the method and system described herein, such as the logic, may be implemented as functionality programmed into any of a variety of circuitry, including programmable logic devices (“PLDs”), such as field programmable gate arrays (“FPGAs”), programmable array logic (“PAL”) devices, electrically programmable logic and memory devices and standard cell-based devices, as well as application specific integrated circuits. Some other possibilities for implementing aspects include: memory devices, microcontrollers with memory (such as 1PROM), embedded microprocessors, firmware, software, etc. Furthermore, aspects may be embodied in microprocessors having software-based circuit emulation, discrete logic (sequential and combinatorial), custom devices, fuzzy (neural) logic, quantum devices, and hybrids of any of the above device types. The underlying device technologies may be provided in a variety of component types, e.g., metal-oxide semiconductor field-effect transistor (“MOSFET”) technologies like complementary metal-oxide semiconductor (“CMOS”), bipolar technologies like emitter-coupled logic (“ECL”), polymer technologies (e.g., silicon-conjugated polymer and metal-conjugated polymer-metal structures), mixed analog and digital, and so on.

The present invention can be embodied in the form of methods and apparatus for practicing those methods. The present invention can also be embodied in the form of program code embodied in tangible media, such as floppy diskettes, CD-ROMs, hard drives, or any other machine-readable storage medium, wherein, when the program code is loaded into and executed by a machine, such as a computer, the machine becomes an apparatus for practicing the invention. The present invention can also be embodied in the form of program code, for example, whether stored in a storage medium, loaded into and/or executed by a machine, or transmitted over some transmission medium, such as over electrical wiring or cabling, through fiber optics, or via electromagnetic radiation, wherein, when the program code is loaded into and executed by a machine, such as a computer, the machine becomes an apparatus for practicing the invention. When implemented on a general-purpose processor, the program code segments combine with the processor to provide a unique device that operates analogously to specific logic circuits.

The software is stored in a machine readable medium that may take many forms, including but not limited to, a tangible storage medium, a carrier wave medium or physical transmission medium. Non-volatile storage media include, for example, optical or magnetic disks, such as any of the storage devices in any computer(s) or the like. Volatile storage media include dynamic memory, such as main memory of such a computer platform. Tangible transmission media include coaxial cables; copper wire and fiber optics, including the wires that comprise a bus within a computer system. Carrier-wave transmission media can take the form of electric or electromagnetic signals, or acoustic or light waves such as those generated during radio frequency (RF) and infrared (IR) data communications. Common forms of computer-readable media therefore include for example: disks (e.g., hard, floppy, flexible) or any other magnetic medium, a CD-ROM, DVD or DVD-ROM, any other optical medium, any other physical storage medium, a RAM, a PROM and EPROM, a FLASH-EPROM, any other memory chip, a carrier wave transporting data or instructions, cables or links transporting such a carrier wave, or any other medium from which a computer can read programming code and/or data. Many of these forms of computer readable media may be involved in carrying one or more sequences of one or more instructions to a processor for execution.

It should also be noted that the various logic and/or functions disclosed herein may be enabled using any number of combinations of hardware, firmware, and/or as data and/or instructions embodied in various machine-readable or computer-readable media, in terms of their behavioral, register transfer, logic component, and/or other characteristics. Computer-readable media in which such formatted data and/or instructions may be embodied include, but are not limited to, non-volatile storage media in various forms (e.g., optical, magnetic or semiconductor storage media) and carrier waves that may be used to transfer such formatted data and/or instructions through wireless, optical, or wired signaling media or any combination thereof. Examples of transfers of such formatted data and/or instructions by carrier waves include, but are not limited to, transfers (uploads, downloads, e-mail, etc.) over the Internet and/or other computer networks by one or more data transfer protocols (e.g., HTTP, FTP, SMTP, and so on).

Unless the context clearly requires otherwise, throughout the description and the claims, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. Additionally, the words “herein,” “hereunder,” “above,” “below,” and words of similar import refer to this application as a whole and not to any particular portions of this application. When the word “or” is used in reference to a list of two or more items, that word covers all of the following interpretations of the word: any of the items in the list, all of the items in the list and any combination of the items in the list.

Although certain presently preferred implementations of the invention have been specifically described herein, it will be apparent to those skilled in the art to which the invention pertains that variations and modifications of the various implementations shown and described herein may be made without departing from the spirit and scope of the invention. Accordingly, it is intended that the invention be limited only to the extent required by the applicable rules of law.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the invention and its practical applications, to thereby enable others skilled in the art to best utilize the invention and various embodiments with various modifications as are suited to the particular use contemplated.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the invention and its practical applications, to thereby enable others skilled in the art to best utilize the invention and various embodiments with various modifications as are suited to the particular use contemplated, etc.

What is claimed is:

1. A method comprising:
by a computer with a processor and a memory in communication with a data storage and a network,
receiving input of a patient demographic data, including maternal age;
causing storage of the patient demographic data;
receiving input of patient historical medical data;
causing storage of the patient historical medical data;
receiving input of patient medical test data;
causing storage of the patient medical test data;
receiving a specific analysis query;
analyzing the patient demographic data, the patient historical medical data, and the patient medical test data for the received specific analysis query;
determining a risk analysis based on the analysis of the received specific analysis query;
sending the risk analysis, corresponding recommendations for follow on care, and new tests;
combining the determined risk analysis with further risk analysis;
plotting the combined risk analysis on a schematic graph;
causing display of the schematic graph of the combined risk analysis in comparison to a mean risk of a general population at the maternal age.

2. The method of claim 1 wherein the determining a risk analysis includes all of the patient medical test data for one particular test, but only a latest test result from a series of another test.

3. The method of claim 1 wherein the new tests are at least one of, NIPT tests and Amniocentesis with CMA analysis.

4. The method of claim 1 wherein the new tests are at least one of, panel carrier testing for known mutations for inherited conditions.

5. The method of claim 1 wherein the patient medical test data is at least one of carrier screening test results for inherited conditions, number of fetuses, nuchal translucency, biochemical markers, ultrasound findings, fetal growth parameters, and parental growth parameters.

6. The method of claim 1 wherein the patient historical medical data includes at least one of, inherited conditions, number of fetuses, nuchal translucency, biochemical markers, ultrasound findings (markers as well as major anomalies/findings), fetal growth parameters as well as parental growth parameters, ultrasound scans, alpha-fetoprotein tests, chorionic callus sampling, amniocentesis, and percutaneous umbilical blood sampling.

7. The method of claim 1 wherein the patient historical medical data includes at least one of, glucose tolerance tests, fetal non-stress tests, biophysical profiles, triple screen tests: to measure low and high levels of AFP, abnormal levels of hCG and estriol, quad screen tests: to measure alpha-fetoprotein (AFP), human chorionic gonadotropin (hCG), Estriol, and Inhibin-A, tests for PAPP-A, Chorionic Villus Sampling (CVS), a urinalysis to analyze blood type, Rh factor, glucose, iron levels, and hemoglobin levels.

8. The method of claim 1 wherein the schematic graph includes a current risk determination graphed against a potential risk analysis if that patient undergoes other tests.

9. The method of claim 1 wherein the schematic graph includes a reduction in risk for each next test.

10. The method of claim 1 wherein the schematic graph includes a selection option to select chromosomal risks and potential reduced risk with additional testing.

11. The method of claim 10 wherein the testing includes at least one of, a calculated risk after non-invasive prenatal testing (NIPT) and a calculated risk after chromosomal microarray (CMA).

12. The method of claim 1 further comprising, causing display of an override results page, displaying a current calculated risk for genetic changes in chromosomal structure.

13. A non-transitory computer-readable medium having computer-executable instructions thereon for a method of test analytics, the method comprising:
receiving input of a patient demographic data, including maternal age;
causing storage of the patient demographic data;
receiving input of patient historical medical data;
causing storage of the patient historical medical data;
receiving input of patient medical test data;
causing storage of the patient medical test data;
receiving a specific analysis query;
analyzing the patient demographic data, the patient historical medical data, and the patient medical test data for the received specific analysis query;
determining a risk analysis based on the analysis of the received specific analysis query;
sending the risk analysis, corresponding recommendations for follow on care, and new tests;
combining the determined risk analysis with further risk analysis;
plotting the combined risk analysis on a schematic graph;
causing display of the schematic graph of the combined risk analysis in comparison to a mean risk of a general population at the maternal age.

14. The non-transitory computer-readable medium of claim 13 wherein the schematic graph includes a current risk determination graphed against a potential risk analysis if that patient undergoes other tests.

15. The non-transitory computer-readable medium of claim 13 wherein the schematic graph includes a reduction in risk for each next test.

16. The non-transitory computer-readable medium of claim 13 wherein the schematic graph includes a selection option to select chromosomal risks and potential reduced risk with additional testing.

17. The non-transitory computer-readable medium of claim 16 wherein the testing includes at least one of, a calculated risk after non-invasive prenatal testing (NIPT) and a calculated risk after chromosomal microarray (CMA).

18. The non-transitory computer-readable medium of claim 13 wherein the method further comprises, causing display of an override results page, displaying a current calculated risk for genetic changes in chromosomal structure.

19. A computer system for analyzing data, comprising:
a processor and a memory in communication with a data storage and a network, the processor configured to, receive and cause storage of a patient demographic data, including maternal age;
receive and cause storage of patient historical medical data;
receive and cause storage of patient medical test data;
analyze the patient demographic data, the patient historical medical data, and the patient medical test data for a specific analysis query;
determine a risk analysis based on the analysis of the received specific analysis query;
combine the determined risk analysis with further risk analysis;
plot the combined risk analysis on a graph;
cause display of the graph of the combined risk analysis in comparison to a mean risk of a general population at the maternal age.

20. The computer system of claim 19 wherein the graph includes a current risk determination graphed against a potential risk analysis if that patient undergoes other tests.
